(19) United States
(12) Patent Application Publication
(10) Pub. No.: US 2008/0171129 A1
(43) Pub. Date: Jul. 17, 2008

(54) DRUG ELUTING MEDICAL DEVICE USING POLYMERIC THERAPEUTICS WITH PATTERNED COATING

(75) Inventors: Shrirang Ranade, Arlington, MA (US); Ascher Shmulewitz, Tel Aviv (IL)

Correspondence Address: RISSMAN JOBSE HENDRICKS & OLIVERIO, LLP, 100 Cambridge Street, Suite 2101, BOSTON, MA, 02114, US

(73) Assignee: Cappella, INC., Auburndale, MA (US)

(21) Appl. No.: 11/839,571

(22) Filed: Aug. 16, 2007

Related U.S. Application Data
(60) Provisional application No. 60/885,097, filed on Jan. 16, 2007.
(60) Provisional application No. 60/885,103, filed on Jan. 16, 2007.
(60) Provisional application No. 60/885,105, filed on Jan. 16, 2007.
(60) Provisional application No. 60/885,112, filed on Jan. 16, 2007.
(60) Provisional application No. 60/885,109, filed on Jan. 16, 2007.
(60) Provisional application No. 60/942,301, filed on Jun. 6, 2007.
(60) Provisional application No. 60/942,309, filed on Jun. 6, 2007.
(60) Provisional application No. 60/943,077, filed on Jun. 11, 2007.

(51) Int. Cl. A61F 2/00; A61K 31/335; B05D 3/00

(52) U.S. Cl. 427/2.3; 424/423; 514/449

(57) ABSTRACT

Disclosed herein are methods of coating an implantable device comprising applying a composition to the device in a predetermined pattern. The composition is then set to form a monolith. The composition applied can comprise a biodegradable polymer linked to a chemical moiety through a covalent bond, wherein the chemical moiety forms a pharmaceutically active agent upon degradation of the covalent bond.

### RELATED APPLICATIONS

This application claims the benefit of priority of U.S. Provisional Application Ser. Nos. 60/885,097, filed Jan. 16, 2007, 60/885,103, filed Jan. 16, 2007, 60/885,105, filed Jan. 16, 2007, 60/885,112, filed Jan. 16, 2007, 60/885,109, filed Jan. 16, 2007, 60/942,301, filed Jun. 6, 2007, 60/942,309, filed Jun. 6, 2007, and 60/943,077, filed Jun. 11, 2007, the disclosures of all of which are incorporated herein by reference in their entirety as if fully set forth herein.

### FIELD OF THE INVENTION

The present invention relates to method of patterning coatings for implantable medical devices.

### BACKGROUND OF THE INVENTION

Restenosis is a complex disease state that is being treated by drug eluting stents (DES). Currently, commercially available DES comprises a coated stent where the coating includes a single drug eluted from a polymeric carrier.

For DES treatments, sustained delivery of the drug is generally desired. With a coating comprising a nonbiodegradable polymeric carrier, the mechanism for drug release into the bloodstream is diffusion of the drug through the polymer. Biodegradable polymers have been developed in stent coatings that ideally should reduce the dependency on diffusion and allow sustained drug delivery due to degradation of the polymer in vivo. However, in practice, even systems employing biodegradable polymers ultimately rely mainly on the diffusion mechanism as the polymer degradation rate is too slow for delivering an effective amount of drug to the bloodstream over the required time period.

Accordingly, there remains a need to develop new coatings for implantable medical devices that allow sustained drug release.

### SUMMARY OF THE INVENTION

One embodiment provides a method of coating a medical device, such as an implantable device, comprising:

applying a composition to the device in a predetermined pattern;

setting the composition to form a monolith,

wherein the composition comprises at least two repeat units, each repeat unit comprising a chemical moiety covalently bonded to least one hydrolysable linking group wherein, the chemical moiety forms a pharmaceutically active agent upon hydrolysis of the covalent bond, and the composition has reduced solubility in an aqueous medium than the free form of the pharmaceutically active agent.

Another embodiment provides a method of coating a medical device, such as an implantable device, comprising:

applying a composition to the device in a predetermined pattern;

setting the composition to form a monolith,

wherein the composition comprises a biodegradable polymer linked to a chemical moiety through a covalent bond, wherein the chemical moiety forms a pharmaceutically active agent upon degradation of the covalent bond.

In one embodiment, the method comprises spray coating.

In one embodiment, the composition is applied as droplets.

In one embodiment, the applying is performed with an inkjet printing device.

In one embodiment, the applying is performed in conjunction with an optical scanner to obtain optical data corresponding with the device.

In one embodiment, the optical data is used to calculate the predetermined pattern.

In one embodiment, setting comprises allowing the composition to dry.

In one embodiment, the composition is less soluble in an aqueous medium than the free form of the pharmaceutically active agent.

In one embodiment, the chemical moiety is covalently bonded to the biodegradable polymer via a linking group.

In one embodiment, the linking group comprises linkages selected from anhydride and ester linkages.

In one embodiment, the chemical moiety is covalently bonded to the biodegradable polymer as a pendant group of the polymer chain.

In one embodiment, the chemical moiety is a portion of the polymer backbone.

In one embodiment, the pharmaceutically active agent is selected from taxanes, limus derivatives, and non-steroidal anti-inflammatory agents.

In one embodiment, the pharmaceutically active agent is selected from paclitaxel, sirolimus, everolimus, and biolimus.

In one embodiment, the biodegradable polymer is present in an amount ranging from 40% to 95% by weight relative to the total weight of the composition.

In one embodiment, the pharmaceutically active agent is present in a dose density ranging from 0.05 to 10 μg/mm².

In one embodiment, the number average molecular weight of the composition is 10,000 Da or less.

In one embodiment, the composition comprises a compound having a Tg greater than 37° C., the compound comprising a biodegradable polymer covalently linked to a chemical moiety of a pharmaceutically active agent, wherein the composition is less in an aqueous medium than the free form of the pharmaceutically active agent.

In one embodiment, the composition comprises at least two repeat units, each repeat unit comprising:

-[D₁-L₁]-

wherein:

L_{1,} is a hydrolysable linking group, and

D₁ is a chemical moiety that upon degradation of covalent bonds binding it to the linking group, forms a pharmaceutically active agent.

In one embodiment, the composition has reduced solubility in an aqueous medium than the free form of the pharmaceutically active agent.

In one embodiment, each repeat unit has the formula:

-[D₁-L₁-BP]-

wherein BP is a biodegradable polymer.

In one embodiment, the composition comprises a polymer comprising the repeat unit:

-[D₁-L₁-D₂-L₂-D₃-L₃-BP-]-

wherein:

L₁, L₂, and L₃ can be the same or different, and each are linking groups capable of covalently bonding to at least one of D₁, D₂, and BP,

D₁ is a chemical moiety that upon degradation of covalent bonds binding it to a linking group and BP, forms an antiproliferative pharmaceutically active agent,

D₂ is a chemical moiety that upon degradation of covalent bonds binding it to linking group, forms an anti-inflammatory agent,

D₃ is a chemical moiety that upon degradation of covalent bonds binding it to linking groups, forms a healing promoter, and

BP is a biodegradable polymer.

In one embodiment, L₁ together with D₁ and D₂, L₂ together with D₁ and D₃, and L₃ together with D₃ and BP, form covalent bonds chosen from anhydride, ester, azo, and carbonate linkages.

In one embodiment, BP is chosen from PGLA and D,L-PLA.

In one embodiment, the composition comprises a polymer comprising the repeat unit:

-[L₂-D₁-L₁-BP-]-

wherein:

L₁ and L₂ can be the same or different, and each are linking groups capable of covalently bonding to at least one of D₁ and BP,

D₁ is a chemical moiety that upon degradation of the polymer, forms a pharmaceutically active agent, and

BP is a biodegradable polymer.

In one embodiment, the composition comprises a polymer comprising the repeat unit:

[L₃-D₁-L₁-D₂-L₂-BP-]-

wherein:

L₁, L₂, and L₃ can be the same or different, and each are linking groups capable of covalently bonding to at least one of D₁, D₂, and BP,

D₁ is a chemical moiety that upon degradation of the polymer, forms a first pharmaceutically active agent,

D₂ is a chemical moiety that upon degradation of the polymer, forms a second pharmaceutically active agent, and

BP is a biodegradable polymer.

In one embodiment, the composition comprises a polymeric material comprising:

a first biodegradable polymer portion comprising a chemical moiety of a pharmaceutically active agent bonded to a spacer group to form a backbone of the first polymer portion;

a second biodegradable polymer portion bonded to the first polymer portion;

wherein the pharmaceutically active agent is bonded to the spacer group via a linkage that is naturally hydrolysable in an in vivo environment, the polymeric material being less soluble in vivo than the free form of the pharmaceutically active agent is soluble in vivo.

In one embodiment, the second polymer material comprises one or more of polyglycolides, polylactides, polycaprolactones, polydioxanones, poly(lactide-co-glycolide), polyhydroxybutyrate, polyhydroxyvalerate, polyphosphoesters, polyphosphoester-urethane, polyamino acids, polycyanoacrylates, poly(trimethylene carbonate), fibrin, fibrinogen, cellulose, starch, collagen, and blends and copolymers of all of the foregoing.

In one embodiment, the composition comprises a polymeric material comprising:

a first biodegradable polymer portion comprising the repeat unit:

[L₃-D₁-L₁-D₂-L₂-]-

wherein:

L₁, L₂, and L₃ can be the same or different, and each are spacer groups capable of covalently bonding to at least one of D₁, D₂ via a linkage that is naturally hydrolysable in vivo;

D₁ is a first chemical moiety that upon hydrolysis of the first polymer portion forms a first pharmaceutically active agent;

D₂ is a second chemical moiety that upon hydrolysis of the first polymer portion forms a second pharmaceutically active agent;

the polymer material further comprising a second biodegradable polymer portion bonded to the first polymer portion.

In one embodiment, the polymeric material is less soluble in vivo than the free form of the first and second pharmaceutically active agents are soluble in vivo.

In one embodiment, the composition comprises a polymeric material comprising:

a first biodegradable polymer portion comprising the repeat unit:

[L₄-D₃-L₃-D₁-L₁-D₂-L₂-]-

wherein:

L₁, L₂, L₃ and L₄ can be the same or different, and each are spacer groups capable of covalently bonding to at least one of D₁, D₂, D₃ via a linkage that is naturally hydrolysable in vivo;

D₁ is a first chemical moiety that upon hydrolysis of the first polymer portion forms a first pharmaceutically active agent;

D₂ is a second chemical moiety that upon hydrolysis of the first polymer portion forms a second pharmaceutically active agent;

D₃ is a third chemical moiety that upon hydrolysis of the first polymer portion forms a third pharmaceutically active agent;

the polymer material further comprising a second biodegradable polymer portion bonded to the first polymer portion.

In one embodiment, the polymeric material is less soluble in vivo than the free form of the first, second and third pharmaceutically active agents are soluble in vivo.

In one embodiment, the device is a stent. In one embodiment, the stent is either balloon expandable or self-expanding.

In one embodiment, the composition is coated on the stent to form a conformal coating around all surfaces of the stent.

In one embodiment, the composition is coated only on the abluminal surface of the stent. In one embodiment, the composition is coated only on the abluminal surface of the stent and the composition resides partially or completely within micro-reservoirs or pores in the stent surface.

In one embodiment, the device is selected from pacemaker leads, valve replacement and repair devices, vena cava filters, and embolic coils and beads.

In one embodiment, the device is an angioplasty balloon having coated thereon the coating comprising the composition, wherein the balloon is used to deliver the composition to an endoluminal surface.

### BRIEF DESCRIPTION OF THE DRAWINGS

Various embodiments of the invention will be understood from the following description, the appended claims and the accompanying drawings, in which:

FIG. 1 is a schematic showing a paclitaxel polymer via a covalent linking group; and

FIG. 2 is a schematic of a multi-layered coating containing different pharmaceutically active agents.

### DETAILED DESCRIPTION

One embodiment provides a method of coating an implantable device comprising:

applying a composition to the device in a predetermined pattern; and

setting the composition to form a monolith.

In one embodiment, the composition comprises at least two repeat units, each repeat unit comprising a chemical moiety covalently bonded to least one hydrolysable linking group wherein, the chemical moiety forms a pharmaceutically active agent upon hydrolysis of the covalent bond, and the composition has reduced solubility in an aqueous medium than the free form of the pharmaceutically active agent.

In another embodiment, the composition comprises a biodegradable polymer linked to a chemical moiety through a covalent bond, wherein the chemical moiety forms a pharmaceutically active agent upon degradation of the covalent bond.

There are numerous methods known in the art for applying coatings. Many of these methods, however, lack the ability to control the precise portions of the device that require the coating. For example, in some instances, to maximize drug delivery, portions of a stent that contact body tissue is coated to deliver a drug to the tissue, whereas there is a lesser need for those portions that do not contact tissue to contain the coating. Moreover, many stents themselves have an intricate scaffolding pattern on which to apply the coating. By applying a predetermined pattern, the process can minimize the use of coating material and maximize the amount of coating used after implantation of the device. Accordingly the present method provides the capability of applying a predetermined pattern to an implantable device, such as a stent.

In one embodiment, the device can be coated partially or wholly with the above defined compositions in any manner known in the art, e.g., spraying, rolling, brushing, electrostatic plating or spinning, vapor deposition (e.g., physical or chemical), air spraying including atomized spray coating, and spray coating using an ultrasonic nozzle. The compositions can be applied by these methods either as a solid (e.g., film or particles), a suspension, a solution, or as a vapor. Alternatively, the device can be coated with a first substance (such as a hydrogel) that is capable of absorbing the composition. In another embodiment, the device can be constructed from a material comprising a polymer/drug composition.

In one embodiment, the method applies a pattern where a substantial portion of the coating covers the portion of the device that contacts body tissue, e.g., a vessel wall. In one embodiment, the portions of the device that do not contact body tissue are substantially free of the coating. The coating can vary in thickness even within a particular predetermined pattern.

In one embodiment, the composition is applied via spray coating. In another embodiment, the composition can be applied by contact coating, e.g., an applicator that applies the composition by patterning the medical device. In another embodiment, the applying is performed with an inkjet printing device in association with an optical scanning device. The scanning device can identify particular features of the medical device, such as edges, different surface types, coordinates, etc. In one embodiment, a computer to obtain and/or process the scanned data of the device and in conjunction with a computer program, apply a desired pattern based on the scanned data. These and other methods for delivering a predetermined pattern are disclosed in U.S. Pat. Nos. 6,645,547, 6,916,379, and 7,048,962, the disclosures of which are incorporated herein by reference.

In one embodiment, the composition is applied as a solution onto a balloon catheter.

After application of the composition, the composition can be set to allow the polymer droplets to coalesce, forming a monolith in the form of the predetermined pattern. In one embodiment, the composition is set by drying, either by air drying, heating, purging with an inert gas, or under reduced pressures.

In one embodiment, the composition to be applied comprises a prodrug for at least one coating covering all or a portion of an implantable medical device. The at least one coating includes a composition comprising a biodegradable polymer linked to a chemical moiety through a covalent bond, where the composition has a Tg greater than 37° C. Upon degradation of the covalent bond, the chemical moiety forms a pharmaceutically active agent.

In one embodiment, the compound has a Tg greater than 40° C. In one embodiment, the compound has a Tg greater than 50° C., or greater than 60° C.

In one embodiment, the composition has reduced solubility in an aqueous medium than the free form of the pharmaceutically active agent. The “free form” of the pharmaceutically active agent can refer to the neutral compound, or salts thereof, e.g., the isolable or stable form of the agent. Thus, the present invention relates to those compositions (comprising the chemical moiety) having a lower solubility in aqueous media (or in physiological media), than the free form of the pharmaceutically active agent. Often during treatment of a disease or condition with a medical device having a coating comprising a drug, the drug is washed away when or after being inserted into a mammal prior to its reaching the target site. In one embodiment, a coating comprising a drug in a form affording it reduced solubility can provide a lesser probability of the drug being inadvertently eliminated by dissolution (or partial dissolution) prior to its reaching the target site.

In one embodiment, a product of the degradation or hydrolysis is the pharmaceutically active agent, e.g., the free form of the agent. In another embodiment, the pharmaceutically active agent has a different structure than the free form of the pharmaceutically active agent but is the true active species that treats the disease or condition, e.g., the form of the agent in vivo.

In one embodiment, a “biodegradable polymer linked to a chemical moiety” refers to a biodegradable polymer linked to a pendant chemical moiety. In another embodiment, a “biodegradable polymer linked to a chemical moiety” refers to a chemical moiety incorporated in the backbone of the polymer.

In one embodiment, the degradation of the covalent bond occurs via hydrolysis. The hydrolysis can involve a direct reaction with an aqueous medium, or can be catalyzed chemically or enzymatically. “Aqueous medium” refers to water, aqueous solutions, physiological media or biological fluids (e.g., body fluids), and other pharmaceutically acceptable media. Suitable hydrolysable covalent bonds include those forming esters, amides, urethanes, carbamates, carbonates, azo linkages, anhydrides, thioesters, and combinations thereof.

In one embodiment, an ester linkage has the formula —OC(═O)—. In one embodiment, a thioester linkage has the formula —SC(═O)—. In one embodiment, an amide linkage has the formula —N(R)C(═O)—, wherein R is a suitable organic radical, such as, for example, hydrogen, (C₁-C₆)alkyl, (C₃-C₆)cycloalkyl, (C₃-C₆)cycloalkyl(C₁-C₆)alkyl, aryl, heteroaryl, aryl(C₁-C₆)alkyl, or heteroaryl(C₁-C₆)alkyl. In one embodiment, a carbamate linkage has the formula —OC(═O)N(R)—, wherein each R is a suitable organic radical as described above. In one embodiment, a “carbonate” linkage has the formula —OC(═O)O—. In one embodiment, an anhydride linkage has the formula —C(═O)—O—C(═O)—. In one embodiment, an azo linkage has the formula —N═N—.

“Biodegradable polymer,” as used herein, refers to a polymer capable of hydrolyzing or otherwise degrading in an aqueous medium. In one embodiment, the resulting product(s) of biodegradation is soluble in the resulting body fluid or, if insoluble, can be suspended in a body fluid and transported away from the implantation site without clogging the flow of the body fluid. The body fluid can be any fluid in the body of a mammal including, but not limited to, blood, serum, urine, saliva, lymph, plasma, gastric, biliary, or intestinal fluids, seminal fluids, and mucosal fluids, humors, and extracellular fluids. In one embodiment, the biodegradable polymer is soluble, degradable as defined above, or is an aggregate of soluble and/or degradable material(s) with insoluble material(s) such that, with the resorption of the soluble and/or degradable materials, the residual insoluble materials are of sufficiently fine size such that they can be suspended in a body fluid and transported away from the implantation site without clogging the flow of the body fluid. Ultimately, the degraded compounds can be eliminated from the body either by excretion in perspiration, urine or feces, or dissolved, degraded, corroded or otherwise metabolized into soluble components that are then excreted from the body.

In one embodiment, the biodegradable polymer imparts at least one mechanical property to the composition, such as adhesion, mechanical integrity, and coating properties. In one embodiment, the biodegradable polymer imparts at least one chemical property, such as chemical stability or reduced solubility in an aqueous medium.

In one embodiment, the biodegradable polymers are degraded through cleavage of functional groups such as esters, anhydrides, carbonates, thioesters, orthoesters, glycosidic bonds, phosphate esters, and amides. Suitable biodegradable polymers include those in the FDA GRAS (Generally Regarded As Safe) list, the disclosure of which is incorporated herein by reference. Exemplary biodegradable polymers include polyglycolides, polylactides (e.g., poly-l-lactide (PLLA)), polycaprolactones, polydioxanones, poly(lactide-co-glycolide) (PLGA), polyhydroxybutyrate, polyhydroxyvalerate, polyphosphoesters, polyphosphoester-urethane, polyamino acids, polycyanoacrylates, poly(trimethylene carbonate), biomolecules such as fibrin, fibrinogen, cellulose, starch, collagen, and blends and copolymers thereof.

In one embodiment, the biodegradable polymer is present in the composition in an amount ranging from 25% to 99% by weight relative to the total weight of the composition, such as an amount ranging from 25% to 95%, from 40% to 99%, or ranging from 40% to 95%.

In one embodiment, the biodegradable polymer comprises PLA in an amount ranging from 15% to 100% by weight relative to the total weight of the biodegradable polymer.

In one embodiment, the biodegradable polymer comprises a blend of polymers. An exemplary blend is PLGA and D,L-PLA. In one embodiment, the biodegradable polymer comprises a blend of 15-85% PLGA, by weight relative to the total weight of the biodegradable polymer, with the remainder PLA.

In one embodiment, the “chemical moiety” is a fragment of a pharmaceutically active agent. For example, upon reacting the pharmaceutically active agent with another species (e.g., a polymer or linker), the portion of the agent that is covalently bonded is the chemical moiety of the agent. A biodegradable polymer “linked to a chemical moiety through a covalent bond” can refer to one or more covalent bonds. Accordingly, in one embodiment, the chemical moiety is linked directly to the polymer via one or more covalent bonds. “Linked directly” as used herein refers to the product of a reaction between the polymer and the pharmaceutically active agent, where the linking atom originates from the starting materials. In another embodiment, the chemical moiety is linked to the biodegradable polymer through covalent bond(s) to a linking group (comprising one or more molecules) or spacer that is covalently bonded to the polymer. Here, the linking group comes from an external reagent and does not originate from either the polymer or the pharmaceutically active agent. Suitable linking groups bind the biodegradable polymer to the chemical moiety through covalent bonds, such as those covalent linkages described herein, e.g., ester, amide, carbamate, carbonate, azo, anhydride, and thioester linkages. Other methods for covalently incorporating pharmaceuticals are provided in Qiu et al., “Polymer Architecture and Drug Delivery,” Pharmaceutical Research, Vol. 23, No. 1, pp. 1-30 (2006), the disclosure of which is incorporated herein by reference.

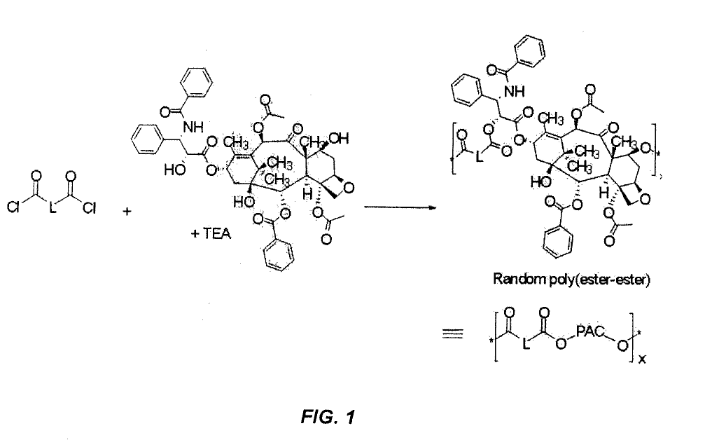

FIG. 1 shows a schematic of a chemical moiety covalently linked to a biodegradable polymer. The chemical moiety of FIG. 1 is paclitaxel (PAC), shown below:

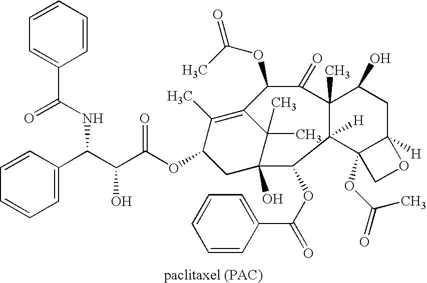

In FIG. 1, a linking group containing two carbonyl chloride functional groups (acyl chlorides if L is, e.g., an alkyl group), is reacted with a hydroxyl group of paclitaxel in the presence of triethylamine (TEA). The resulting —[C(═O)—L—C(═O)—O—PAC—O]— unit can be covalently bonded to a biodegradable polymer via its residual carbonyl chloride group or via a subsequently introduced second linker group. In another embodiment, the L is a biodegradable polymer, resulting in the paclitaxel being directly bonded to the polymer. In either embodiment, the paclitaxel is bonded to the polymer via a series of carbonate/ester linkages, and other linkages such as anhydride, carbamate, etc., depending on the linking group and polymers.

In one embodiment, more than one pharmaceutically active agent other than the agent covalently bonded can be incorporated in the polymer. The additional agents can be either covalently bonded to the polymer or even admixed with the polymer, so long as at least one agent is covalently bonded to the polymer.

In one embodiment, the linking group can impart mechanical properties and release kinetics for the selected therapeutic application. In one embodiment, the linking group is a divalent organic radical having a molecular weight ranging from 25 daltons to 400 daltons, e.g., a molecular weight ranging from 40 daltons to 200 daltons.

In one embodiment, the linking group has a length ranging from 5 angstroms to 100 angstroms using standard bond lengths and angles, e.g., a length ranging from 10 angstroms to 50 angstroms.

The linking group may be biologically inactive, or may itself possess biological activity. The linking group can also comprise other functional groups (including hydroxy groups, mercapto groups, amine groups, carboxylic acids, as well as others) that can be used to modify the properties of the polymer (e.g. for branching, for cross linking, for appending other molecules (e.g. another biologically active compound) to the polymer, for reducing the solubility of the polymer, or for effecting the biodistribution of the polymer).

In one embodiment, the linking group is: a (C₁-C₆)alkyl, e.g., methyl, ethyl, propyl, isopropyl, butyl, isobutyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

In one embodiment, the linking group is a divalent, branched or unbranched, saturated or unsaturated, hydrocarbon chain, having from 1 to 20 carbon atoms, wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

In another embodiment, the linking group is a divalent, branched or unbranched, saturated or unsaturated, hydrocarbon chain, having from 1 to 20 carbon atoms, wherein one or more (e.g. 1, 2, 3, or 4) of the carbon atoms is optionally replaced by (—O—) or (—NR—).

In another embodiment, the linking group is a divalent, branched or unbranched, saturated or unsaturated, hydrocarbon chain, having from 3 to 15 carbon atoms, wherein one or more (e.g. 1, 2, 3, or 4) of the carbon atoms is optionally replaced by (—O—) or NR—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

In another embodiment, the linking group is a divalent, branched or unbranched, saturated or unsaturated, hydrocarbon chain, having from 3 to 15 carbon atoms, wherein one or more (e.g. 1, 2, 3, or 4) of the carbon atoms is optionally replaced by (—O—) or (—NR—).

Other linking groups are disclosed in U.S. Pat. Nos. 6,613,807, and 6,685,928, and U.S. Patent Publication Nos. 20060188546 and 20050031577, the disclosures of which are incorporated herein by reference.

In another embodiment, the linking group is selected from amino acids and peptides.

In one embodiment, the linking group is present in an amount ranging from 5% to 50% by weight relative to the total weight of the composition.

Exemplary pharmaceutically active agents include antiproliferative agents (e.g., those active against smooth muscle cells), anti-inflammatory agents, and healing promoters. Exemplary antiproliferative agents include paclitaxel, sirolimus, everolimus, biolimus, zotarolimus, AP23573 (a sirolimus analog), and other limus derivatives. Exemplary anti-inflammatory agents include non-steroidal agents (e.g., 3-amino-4-hydroxybutyric acid, aceclofenac, alminoprofen, bromfenac, bumadizon, carprofen, diclofenac, diflunisal, enfenamic acid, etodolac, fendosal, flufenamic acid, gentisic acid, meclofenamic acid, mefenamic acid, mesalamine, niflumic acid, olsalazine oxaceprol, S-adenosylmethionine, salicylic acid, salsalate, sulfasalazine, tolfenamic acid). Exemplary healing promoters include nitric oxide donors such as halofuganone, S-nitrosothiols, and glyceryl trinitrite 1-[N-(3-aminopropyl)—N-(3-ammoniopropyl]diazen-1-ium-1,2-diolate, 1-[N-(2-aminoethyl)—N-(2-ammonioethyl)amino]diazen-1-ium-1,2-diolate, as well as epidermal growth factor and other growth factors.

Other exemplary pharmaceutically active agents include analgesics, anesthetics, anti acne agents, antibiotics, synthetic antibacterial agents, anticholinergics, anticoagulants, antidyskinetics, antifibrotics, antifungal agents, antiglaucoma agents, anti-inflammatory agents, antineoplastics, antiosteoporotics, antipagetics, anti-Parkinson's agents, antisporatics, antipyretics, antiseptics/disinfectants, antithrombotics, bone resorption inhibitors, calcium regulators, keratolytics, sclerosing agents and ultraviolet screening agents. Exemplary antithrombotics and anticoagulants include aspirin and plavix.

In one embodiment, the pharmaceutically active agent is a drug useful for treating diseases and conditions associated with restenosis, e.g., antithrombotics, anticoagulants, antiplatelet agents, thrombolytics, antiproliferatives, anti-inflammatories, antimitotic, antimicrobial, agents that inhibit restenosis, smooth muscle cell inhibitors, antibiotics, fibrinolytic, immunosuppressive, and anti-antigenic agents.

Examples of anti-bacterial compounds suitable for use in the present invention include, but are not limited to, 4-sulfanilamidosalicylic acid, acediasulfone, amfenac, amoxicillin, ampicillin, apalcillin, apicycline, aspoxicillin, aztreonam, bambermycin(s), biapenem, carbenicillin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, ciprofloxacin, clinafloxacin, cyclacillin, enoxacin, epicillin, flomoxef, grepafloxacin, hetacillin, imipenem, lomefloxacin, lymecycline, meropenem, moxalactam, mupirocin, nadifloxacin, norfloxacin, panipenem, pazufloxacin, penicillin N, pipemidic acid, quinacillin, ritipenem, salazosulfadimidine, sparfloxacin, succisulfone, sulfachrysoidine, sulfaloxic acid, teicoplanin, temafloxacin, temocillin, ticarcillin, tigemonam, tosufloxacin, trovafloxacin, and vancomycin.

Examples of anti-fungal compounds suitable for use in the present invention include, but are not limited to amphotericin B, azaserine, candicidin(s), lucensomycin, natamycin, and nystatin.

Examples of anti-neoplastic compounds suitable for use in the present invention include, but are not limited to 6-diazo-5-oxo-L-norleucine, azaserine, carzinophillin A, denopterin, edatrexate, eflomithine, melphalan, methotrexate, mycophenolic acid, podophyllinic acid 2-ethylhydrazide, pteropterin, streptonigrin, Tomudex. RTM. (N-((5-(((1,4-Dihydro-2-methyl-4-oxo-6-quinazolinyl)methyl)methylamino)-2-thienyl)carbonyl)-L-glutamic acid), and ubenimex.

Examples of anti-thrombotic compounds for use in the present invention include, but are not limited to, argatroban, iloprost, lamifiban, taprostene, and tirofiban.

Examples of immunosuppressive compounds suitable for use in the present invention include, but are not limited to bucillamine, mycophenolic acid, procodazole, romurtide, and ubenimex.

Dosages of the pharmaceutically active agent may be determined by means known in the art. Typically, the dosage is dependent upon the particular drug employed and medical condition being treated to achieve a therapeutic result. In one embodiment, the amount of drug represents about 0.001 percent to about seventy percent of the total coating weight, or about 0.01 percent to about sixty percent of the total coating weight. In one embodiment, the weight percent of the therapeutic agents in the carrier or polymer coating is 1% to 50%, 2% to 45%, 5% to 40%, or 10% to 25% by weight relative to the total coating weight. In another embodiment, it is possible that the drug may represent as little as 0.0001 percent to the total coating weight. In another embodiment, the dosage is determined per coated surface area of the device. For example, the dose density may range from 0.05 to 10 μg/mm², such as a dose-density ranging from 0.05 to 1.0 μg/mm², or ranging from 0.1 to 4 μg/mm², or ranging from 0.2 to 4 μg/mm².

In one embodiment, the device delivers the agent over a selected period of time, such as days, weeks or months, e.g., such as a period of at least one week, at least two weeks, at least one month, at least six months, or at least one year.

In one embodiment, the number average molecular weight of the composition is 20,000 Da or less, such as a number average molecular weight of 10,000 Da or less.

Another embodiment provides a polymeric material comprising:

a first biodegradable polymer portion comprising a chemical moiety of a pharmaceutically active agent bonded to a spacer group to form a backbone of the first polymer portion;

a second biodegradable polymer portion bonded to the first polymer portion;

wherein the pharmaceutically active agent is bonded to the spacer group via a linkage that is naturally hydrolysable in an in vivo environment, the polymeric material being less soluble in vivo than the free form of the pharmaceutically active agent is soluble in vivo.

In one embodiment, the at least one pharmaceutically active agent is hydrophobic or amphipathic (e.g., paclitaxel). Although hydrophobic agents may have some solubility in water, generally a hydrophobic agent generally dissolves more readily in oils or non-polar solvents than in water or polar solvents. In one embodiment, the agent is hydrophilic, e.g., dissolves more readily in water or polar solvents than in oils or non-polar solvents.

In another embodiment, the composition comprises a polymer comprising the repeat unit:

-[D₁-L₁-D₂-L₂-D₃-L₃-BP]-

wherein:

L₁, L₂, and L₃ can be the same or different and are linking groups,

D₁ is a chemical moiety that upon degradation of covalent bonds binding it to a linking group and BP, forms an antiproliferative pharmaceutically active agent,

D₂ is a chemical moiety that upon degradation of covalent bonds binding it to linking groups, forms an anti-inflammatory agent,

D₃ is a chemical moiety that upon degradation of covalent bonds binding it to linking groups, forms a healing promoter, and

BP is a biodegradable polymer, such as the polymers disclosed herein.

In one embodiment, the polymer is less soluble in an aqueous medium (e.g., a physiological medium) than is the free form of any of the pharmaceutically active agents.

In this embodiment, various drugs are incorporated in the polymer to impart different therapeutic effects. The choice of L₁, L₂, L₃, and the biodegradable polymer can allow control of release profile and kinetics of pharmaceutically active agents from the medical device. For example, the release profile and kinetics can be controlled by the hydrolysis rates and chemistry of the various hydrolytic linkages. The period of time of drug delivery and drug dosage can be controlled to substantially prevent undesirable burst release. Moreover, the linking groups and biodegradable polymer can be chosen to provide desirable mechanical properties.

In one embodiment, a “polymer comprising the repeat unit” can have additional linking groups and repeat units other than the repeat unit listed herein.

In another embodiment, pharmaceutically active agents in addition to D₁, D₂, and D₃ can also be present in the composition, e.g., any other agents useful for treating vascular injury, e.g., restenosis. Alternatively, pharmaceutically active agents in addition to D₁, D₂, and D₃ can be incorporated in the polymer, e.g., either covalently linked to the polymer or admixed with the polymer.

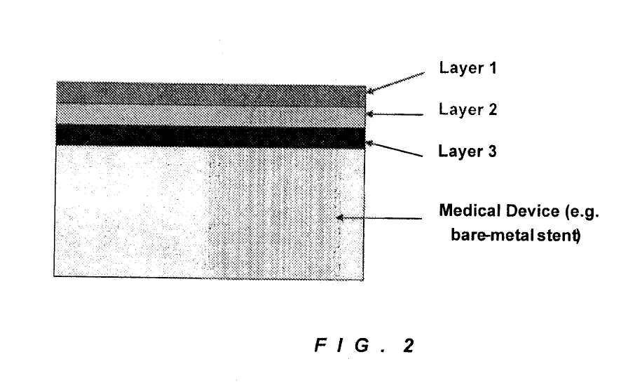

In one embodiment, two or more coatings are applied to the device where each coating contains a different pharmaceutically active agent. FIG. 2 is a schematic showing a multi-layered coating arrangement, where each of layers 1, 2, and 3 contain either a unique pharmaceutically active agent, or if two or more layers contain the same agent, the agent is linked to the polymer via a different linking chemistry. This arrangement allows control of the release profile of the agents and can provide control of the sequence of release of different pharmaceutically active agents. In one embodiment, each layer can be individually customized by choice of agents, linking chemistry, polymer structure, thickness, etc. for controlling the release profile and kinetics.

In one embodiment, each layer contains a unique agent, e.g., D₁, D₂, and D₃, as described herein, or any other agents useful for treating vascular injury, e.g., restenosis. Alternatively, pharmaceutically active agents in addition to D₁, D₂, and D₃ can be incorporated in the polymer, e.g., either covalently linked to the polymer or admixed with the polymer.

In one embodiment, the device treats narrowing or obstruction of a body passageway in a subject in need thereof. In another embodiment, the method comprises inserting the device into the passageway, the device comprising a generally tubular structure, the surface of the structure being coated with a composition disclosed herein, such that the passageway is expanded. In the method, the body passageway may be selected from arteries, veins, lacrimal ducts, trachea, bronchi, bronchiole, nasal passages, sinuses, eustachian tubes, the external auditory canal, oral cavities, the esophagus, the stomach, the duodenum, the small intestine, the large intestine, biliary tracts, the ureter, the bladder, the urethra, the fallopian tubes, uterus, vagina, the vasdeferens, and the ventricular system.

In one embodiment, the implantable devices disclosed herein are implanted in a subject in need thereof to achieve a therapeutic effect, e.g., therapeutic treatment and/or prophylactic/preventative measures. Those in need of treatment may include individuals already having a particular medical disease as well as those at risk for the disease (e.g., those who are likely to ultimately acquire the disorder). A therapeutic method can also result in the prevention or amelioration of symptoms, or an otherwise desired biological outcome, and may be evaluated by improved clinical signs, delayed onset of disease, reduced/elevated levels of lymphocytes and/or antibodies.

In one embodiment, the method is used for treating at least one disease or condition associated with vascular injury or angioplasty, e.g., one or more of atherosclerosis, restenosis, neointima, neointimal hyperplasia and thrombosis.

Exemplary devices include sutures, staples, anastomosis devices, vertebral disks, bone pins, suture anchors, hemostatic barriers, clamps, screws, plates, clips, vascular implants, urological implants, tissue adhesives and sealants, tissue scaffolds, bone substitutes, intraluminal devices, and vascular supports. For example, the device can be a cardiovascular device, such as venous catheters, venous ports, tunneled venous catheters, chronic infusion lines or ports, including hepatic artery infusion catheters, pacemakers and pace maker leads, and implantable defibrillators. Alternatively, the device can be a neurologic/neurosurgical device such as ventricular peritoneal shunts, ventricular atrial shunts, nerve stimulator devices, dural patches and implants to prevent epidural fibrosis post-laminectomy, and devices for continuous subarachnoid infusions. The device can be a gastrointestinal device, such as chronic indwelling catheters, feeding tubes, portosystemic shunts, shunts for ascites, peritoneal implants for drug delivery, peritoneal dialysis catheters, and suspensions or solid implants to prevent surgical adhesions. In another example, the device can be a genitourinary device, such as uterine implants, including intrauterine devices (IUDs) and devices to prevent endometrial hyperplasia, fallopian tubal implants, including reversible sterilization devices, fallopian tubal stents, artificial sphincters and periurethral implants for incontinence, ureteric stents, chronic indwelling catheters, bladder augmentations, or wraps or splints for vasovasostomy, central venous catheters.

Other exemplary devices include prosthetic heart valves, vascular grafts ophthalmologic implants (e.g., multino implants and other implants for neovascular glaucoma, drug eluting contact lenses for pterygiums, splints for failed dacrocystalrhinostomy, drug eluting contact lenses for corneal neovascularity, implants for diabetic retinopathy, drug eluting contact lenses for high risk corneal transplants), otolaryngology devices (e.g., ossicular implants, Eustachian tube splints or stents for glue ear or chronic otitis as an alternative to transtempanic drains), plastic surgery implants (e.g., breast implants or chin implants), and catheter cuffs and orthopedic implants (e.g., cemented orthopedic prostheses).

In one embodiment, the device is selected from pacemaker leads, valve replacement and repair devices, vena cava filters, and embolic coils and beads.

Another exemplary device according to the invention is a stent, such as a stent comprising a generally tubular structure. A stent is commonly used as a tubular structure disposed inside the lumen of a duct to relieve an obstruction. In one embodiment, the stent is either balloon expandable or self-expanding. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen.

An exemplary stent is a stent for treating narrowing or obstruction of a body passageway in a human or animal in need thereof. “Body passageway” as used herein refers to any of number of passageways, tubes, pipes, tracts, canals, sinuses or conduits which have an inner lumen and allow the flow of materials within the body. Representative examples of body passageways include arteries and veins, lacrimal ducts, the trachea, bronchi, bronchiole, nasal passages (including the sinuses) and other airways, eustachian tubes, the external auditory canal, oral cavities, the esophagus, the stomach, the duodenum, the small intestine, the large intestine, biliary tracts, the ureter, the bladder, the urethra, the fallopian tubes, uterus, vagina and other passageways of the female reproductive tract, the vasdeferens and other passageways of the male reproductive tract, and the ventricular system (cerebrospinal fluid) of the brain and the spinal cord. Exemplary devices of the invention are for these above-mentioned body passageways, such as stents, e.g., vascular stents. There is a multiplicity of different vascular stents known in the art that may be utilized following percutaneous transluminal coronary angioplasty.

Any number of stents may be utilized in accordance with the present invention and the invention is not limited to the specific stents that are described in exemplary embodiments of the present invention. The skilled artisan will recognize that any number of stents may be utilized in connection with the present invention. In addition, as stated above, other medical devices may be utilized, such as e.g., orthopedic implants.

In one embodiment, the composition is coated on the stent to form a conformal coating around all surfaces of the stent. In another embodiment, the composition is coated only on the abluminal surface of the stent. In one embodiment, the composition resides partially or completely within micro-reservoirs or pores in the stent surface.

In one embodiment, the device is an angioplasty balloon having coated thereon the coating comprising the composition, wherein the balloon is used to deliver the composition to an endoluminal surface.

What is claimed is:

1. A method of coating a medical device, such as an implantable device, comprising:
applying a composition to the device in a predetermined pattern;
setting the composition to form a monolith,
wherein the composition comprises a biodegradable polymer linked to a chemical moiety through a covalent bond, wherein the chemical moiety forms a pharmaceutically active agent upon degradation of the covalent bond.

2. The method of claim 1 wherein the step of applying comprises spray coating or applying the composition as droplets or applying the composition with an inkjet printing device.

3. The method of claim 1 wherein the step of applying is performed in conjunction with an optical scanner to obtain optical data corresponding with the device, the optical data being used to calculate the predetermined pattern.

4. The method of claim 1 wherein the step of setting comprises allowing the composition to dry.

5. The method of claim 1 wherein the composition is less soluble in an aqueous medium than the free form of the pharmaceutically active agent.

6. The method of claim 1 wherein the chemical moiety is covalently bonded to the biodegradable polymer via a linking group.

7. The method of claim 6 wherein the linking group comprises anhydride or ester linkages.

8. The method of claim 1 wherein the chemical moiety is covalently linked to the biodegradable polymer as a pendant group to a polymer chain.

9. The method of claim 1 wherein the chemical moiety is a portion of the polymer backbone.

10. The method of claim 1 wherein the pharmaceutically active agent is selected from taxanes, limus derivatives, and non-steroidal anti-inflammatory agents.

11. The method of claim 1 wherein the pharmaceutically active agent is selected from paclitaxel, sirolimus, everolimus, and biolimus.

12. The method of claim 1 wherein the biodegradable polymer is present in an amount ranging from 40% to 95% by weight relative to the total weight of the composition.

13. The method of claim 1 wherein the pharmaceutically active agent is present in a dose density ranging from about 0.05 to about 10 μg/mm².

14. The method of claim 1 wherein the number average molecular weight of the composition is 10,000 Da or less.

15. The method of claim 1 wherein the composition comprises a compound having a Tg greater than 37° C., the compound comprising a biodegradable polymer covalently linked to a chemical moiety of a pharmaceutically active agent, wherein the composition on biodegradation is less soluble in an aqueous medium than the free form of the pharmaceutically active agent.

16. The method of claim 2 wherein the step of applying is performed in conjunction with an optical scanner to obtain optical data corresponding with the device, the optical data being used to calculate the predetermined pattern.

17. The method of claim 16 wherein the step of setting comprises allowing the composition to dry.

18. The method of claim 1 wherein the composition comprises at least two repeat units, each repeat unit comprising:
-[D₁-L₁]-
wherein:
L₁ is a hydrolysable linking group, and
D₁ is a chemical moiety that upon degradation of all covalent bonds that bond D₁ to the linking group, forms a pharmaceutically active agent.

19. The method of claim 1 wherein the composition comprises at least two repeat units, each repeat unit comprising one or the other of:
-[D₁-L₁-BP]- and -[L₁-D₁-BP]-
wherein:
L₁ is a hydrolysable linking group,
D₁ is a chemical moiety that upon degradation of all covalent bonds that bond D₁ to the linking group and the BP, forms a pharmaceutically active agent, and
BP is a biodegradable polymer.

20. The method of claim 1 wherein the composition comprises a polymer comprising the repeat unit:
-[D₁-L₁-D₂-L₂-D₃-L₃-BP-]-
wherein:
L₁, L₂, and L₃ can be the same or different, and each are linking groups capable of covalently bonding to at least one of D₁, D₂, and BP,
D₁ is a chemical moiety that upon degradation of all covalent bonds binding it to an adjacent group, forms an antiproliferative pharmaceutically active agent,
D₂ is a chemical moiety that upon degradation of covalent bonds binding it to linking group, forms an anti-inflammatory agent,
D₃ is a chemical moiety that upon degradation of covalent bonds binding it to linking groups, forms a healing promoter, and
BP is a biodegradable polymer.

21. The method of claim 20 wherein L₁ together with D₁ and D₂, L₂ together with together with D₁ and D₃, and L₃ together with D₃ and BP, form covalent bonds chosen from anhydride, ester, azo, and carbonate linkages.

22. The method of claim 19 wherein BP is chosen from PLGA and D,L-PLA.

23. The method of claim 1 wherein the composition comprises a polymer comprising the repeat unit:
-[L₂-D₁-L₁-BP-]-
wherein:
L₁ and L₂ can be the same or different, and each are linking groups capable of covalently bonding to an adjacent D₁ or BP,
D₁ is a chemical moiety that upon degradation of the polymer, forms a pharmaceutically active agent, and
BP is a biodegradable polymer.

24. The method of claim 1 wherein the composition comprises a polymer comprising the repeat unit:
[L₃-D₁-L₁-D₂-L₂-BP-]-
wherein:
L₁, L₂, and L₃ can be the same or different, and each are linking groups capable of covalently bonding to an adjacent D₁, D₂, and BP,
D₁ is a chemical moiety that upon degradation of the polymer, forms a first pharmaceutically active agent,
D₂ is a chemical moiety that upon degradation of the polymer, forms a second pharmaceutically active agent, and
BP is a biodegradable polymer.

25. The method of claim 1 wherein the composition comprises a polymeric material comprising:
a first biodegradable polymer portion comprising a chemical moiety of a pharmaceutically active agent bonded to a spacer group to form a backbone of the first polymer portion;
a second biodegradable polymer portion bonded to the first polymer portion;
wherein the pharmaceutically active agent is bonded to the spacer group via a linkage that is naturally hydrolysable in an in vivo environment, the polymeric material being less soluble in vivo than the free form of the pharmaceutically active agent is soluble in vivo.

26. The method of claim 25 wherein the second polymer portion comprises one or more of polyglycolides, polylactides, polycaprolactones, polydioxanones, poly(lactide-co-glycolide), polyhydroxybutyrate, polyhydroxyvalerate, polyphosphoesters, polyphosphoester-urethane, polyamino acids, polycyanoacrylates, poly(trimethylene carbonate), fibrin, fibrinogen, cellulose, starch, collagen, and blends and copolymers of all of the foregoing.

27. The method of claim 1 wherein the composition comprises a polymeric material comprising:
a first biodegradable polymer portion comprising the repeat unit:
[L₃-D₁-L₁-D₂-L₂-]-
wherein:
L₁, L₂, and L₃ can be the same or different, and each are spacer groups capable of covalently bonding to an adjacent D₁, D₂ via a linkage that is naturally hydrolysable in vivo;
D₁ is a first chemical moiety that upon hydrolysis of the first polymer portion forms a first pharmaceutically active agent;
D₂ is a second chemical moiety that upon hydrolysis of the first polymer portion forms a second pharmaceutically active agent;
the polymer material further comprising a second biodegradable polymer portion bonded to the first polymer portion.

28. The method of claim 27 wherein the polymeric material is less soluble in vivo than the free form of the first and second pharmaceutically active agents are soluble in vivo.

29. The method of claim 1 wherein the composition comprises a polymeric material comprising:
a first biodegradable polymer portion comprising the repeat unit:
[L₄-D₃-L₃-D₁-L₁-D₂-L₂-]-
wherein:
L₁, L₂, L₃ and L₄ can be the same or different, and each are spacer groups capable of covalently bonding to an adjacent D₁, D₂, D₃ via a linkage that is naturally hydrolysable in vivo;
D₁ is a first chemical moiety that upon hydrolysis of the first polymer portion forms a first pharmaceutically active agent;
D₂ is a second chemical moiety that upon hydrolysis of the first polymer portion forms a second pharmaceutically active agent;
D₃ is a third chemical moiety that upon hydrolysis of the first polymer portion forms a third pharmaceutically active agent;
the polymer material further comprising a second biodegradable polymer portion bonded to the first polymer portion.

30. The method of claim 29 wherein the polymeric material is less soluble in vivo than the free form of the first, second and third pharmaceutically active agents are soluble in vivo.

31. The method of claim 1 wherein the device is a stent that is either balloon expandable or self-expanding.

32. The method of claim 31 wherein the composition is coated on the stent to form a conformal coating around all surfaces of the stent.

33. The method of claim 31 wherein the composition is coated only on an abluminal surface of the stent.

34. The method of claim 33 wherein the composition resides partially or completely within micro-reservoirs or pores in the stent surface.

35. The method of claim 1 wherein the device is selected from pacemaker leads, valve replacement and repair devices, vena cava filters, and embolic coils and beads.

36. The method of claim 1 wherein the device is an angioplasty balloon having coated thereon the coating comprising the composition, wherein the balloon is used to deliver the composition to an endoluminal surface.

37. A composition comprising at least two repeat units, each repeat unit comprising one or the other of:
-[D₁-L₁-BP]- and -[L₁-D₁-BP]-
wherein:
L₁ is a hydrolysable linking group,
D₁ is a chemical moiety that upon degradation of all covalent bonds that bond D₁ to the linking group and the BP, forms a pharmaceutically active agent, and
BP is a biodegradable polymer.

38. The composition of claim 37 wherein:
L₁ is one or more of the following linkages or is a moiety that is linked to an adjacent BP or D₁ by one or the other of the following linkages: esters, amides, urethanes, carbamates, carbonates, azo, anhydrides, thioesters.

39. The composition of claim 37 wherein BP is selected from polyglycolides, polylactides (e.g., poly-l-lactide (PLLA)), polycaprolactones, polydioxanones, poly(lactide-co-glycolide) (PLGA), polyhydroxybutyrate, polyhydroxyvalerate, polyphosphoesters, polyphosphoester-urethane, polyamino acids, polycyanoacrylates, poly(trimethylene carbonate), biomolecules such as fibrin, fibrinogen, cellulose, starch, collagen, and blends and copolymers of all of the foregoing.

40. The composition of claim 37 wherein D₁ is selected from taxanes, limus derivatives, and non-steroidal anti-inflammatory agents.

41. The composition of claim 37 wherein the pharmaceutically active agent is selected from paclitaxel, sirolimus, everolimus, biolimus and derivatives and analogs of all of the foregoing.
